(19) United States
(12) Patent Application Publication
(10) Pub. No.: US 2026/0132434 A1
(43) Pub. Date: May 14, 2026

(54) ENZYMATIC SYNTHESIS OF D-ALLULOSE AND ITS DERIVATIVES FROM FUSION ENZYMES

(71) Applicant: FERTIS INDIA PVT LTD, Hyderabad (IN)

(72) Inventor: Rahul Raju Kanumuru, Hyderabad (IN)

(21) Appl. No.: 18/875,633

(22) PCT Filed: Jun. 16, 2023
(86) PCT No.: PCT/IN2023/050576

§ 371 Date: Dec. 16, 2024

(30) Foreign Application Priority Data
Jun. 16, 2022 (IN) 202141058821

(51) Int. Cl. C12P 19/02; C12N 9/12; C12N 9/16; C12N 9/90; C12P 19/24

(52) U.S. Cl. CPC C12P 19/02; C12N 9/1205; C12N 9/16; C12N 9/90; C12P 19/24; C12Y 207/01004; C07K 2319/00

(57) ABSTRACT

The present invention belongs to the field for enzymatic catalysis, particularly relates to the usage of multienzyme system/fusion proteins for producing of D-allulose, methods for production of the fusion enzyme and their use thereof for the production of D-allulose and its derivatives from carbohydrate sources. More particularly, the present invention provides simple, improved and cost-effective process for the production of D-allulose and its derivatives from carbohydrate sources from such fusion enzymes.

### FIELD OF INVENTION

The present invention belongs to the field for enzymatic catalysis, particularly relates to the usage of fusion enzyme system for producing of D-allulose, methods for production of the fusion enzyme and their use thereof for the production of D-allulose.

### BACKGROUND ART

Allulose is also known as D-psicose is a low-calorie, natural sweetener that has 70% the sweetness of sucrose, but only 10% of the calories. It is a naturally occurring monosaccharide and present in small quantities in Wheat and other plants, hence also classified under “Rare Sugars”. Rare sugars are a kind of monosaccharides and their derivatives that rarely exist in nature (as defined by the International Society of Rare Sugars (ISRS) in 2002). In recent years, D-allulose, an epimer of fructose, has attracted wide attention in the fields of diet, health care, medicine, etc. D-allulose has 70% of the sweetness of sucrose, but the energy value of D-allulose is only 0.007 kcal/g, and D-allulose has only 0.3% efficiency of energy deposition of sucrose. Therefore, D-allulose is an ideal low-calorie sweetener, and can be used as a sucrose substitute in food applications; D-allulose has been proven to have an anti-hypoglycaemic effect, and also inhibit the activities of hepatic fatty synthase and intestinal α-glucosidase thus reducing the abdominal fat deposition, and also has high medical value in the treatment of neurodegenerative and atherosclerotic diseases. It has several health benefits: close to zero calorie, very low glycaemic index of 1, it helps regulate the blood sugar etc.

Conventionally, D-allulose is produced from fructose using D-allulose 3-epimerase as the catalyst. The conversion rate of this enzymatic conversion is low, and the highest conversion is only 32% and this process requires a simulated moving bed chromatography system to realize the recycling of fructose which increases the production cost.

The US patent 20210254031A1, describes a process the production of D-allulose using recombinant cells or multienzyme cascade system for the production of D-allulose from starch or sucrose or other carbohydrates. This process requires several enzymes that have to be used in single pot to convert sucrose to D-allulose. One of the major disadvantages is the conversion of fructose to glucose using glucose isomerase. This conversion step has been reported to have an efficiency of only 30% therefore the separation of glucose from the reaction mixture is essential for maintaining the forward reaction. Moreover, several enzymes with different optimal conditions are used in single pot, thereby operating some conversion in suboptimal conditions.

The U.S. Pate. No. 11,053,528B2, discloses a process for the production of fructose 6 phosphate from sucrose using multiple enzymes like sucrose phosphorylase, phosphoglucomutase, glucose phosphate isomerase and glucose isomerase. In this process the conversion of fructose to glucose is not favoured and has been reported to have an efficiency of only 30%. The process needs to be highly regulated to achieve economical and optimal conversion.

Therefore, there exists a need for an alternative cost-effective approach for high yield allulose production. Ideally, this pathway should incorporate energetically favourable chemical reactions at least in one step of the process. Additionally, it is desirable to have a production process that can be conducted in a single tank or bioreactor. Furthermore, a production method that can operate at a relatively low phosphate concentration, with the potential for phosphate recycling, would be beneficial. Additionally, it would be advantageous to have an allulose production pathway that does not rely on the expensive nicotinamide adenosine dinucleotide (NAD(H)) coenzyme in any of the reaction steps.

Thus, the inventors of the present invention have developed a novel process for the production of D-allulose and its derivatives that is simple, cost-effective and has high conversion rate that addresses all the concerns and limitations of the prior art.

### SUMMARY OF THE PRESENT INVENTION

The present invention relates to a process for the production of D-allulose and its derivatives from sucrose or fructose or glucose derived from starch and/or cellulose. The present invention provides a simple process for the production of D-allulose, fructose, allose and allitol wherein the claimed process overcomes the equilibrium limitations of conventional isomerization and epimerization processes and results in higher conversion efficiencies.

Further, the present invention relates to fusion proteins expressing one or more enzymes for the conversion of carbohydrate sources to D-allulose and to processes for the production of such fusion proteins.

### BRIEF DESCRIPTION OF THE FIGURES

The accompanying drawings illustrate some of the embodiments of the present invention and together with the descriptions, serve to explain the invention. These drawings have been provided by way of illustration and not by way of limitation. The components in the drawings are not necessarily drawn to scale, emphasis instead being placed upon clearly illustrating the principles of the aspects of the embodiments.

FIG. 1: Schematic representation of a process for the production of D-allulose and its derivatives from sucrose or fructose or glucose derived from starch and/or cellulose.

FIG. 2: SHOWS THE SDS-PAGE ANALYSIS OF FRUCTOKINASE

FIG. 3: shows the SDS-PAGE analysis of Allulose 6-phosphate epimerase

FIG. 4: shows the SDS-PAGE analysis of Allulose 6-phosphate phosphatase

FIG. 5: shows the SDS-PAGE analysis of Allulose 6-phosphate epimerase-Allulose 6-phosphate phosphatase Fusion protein.

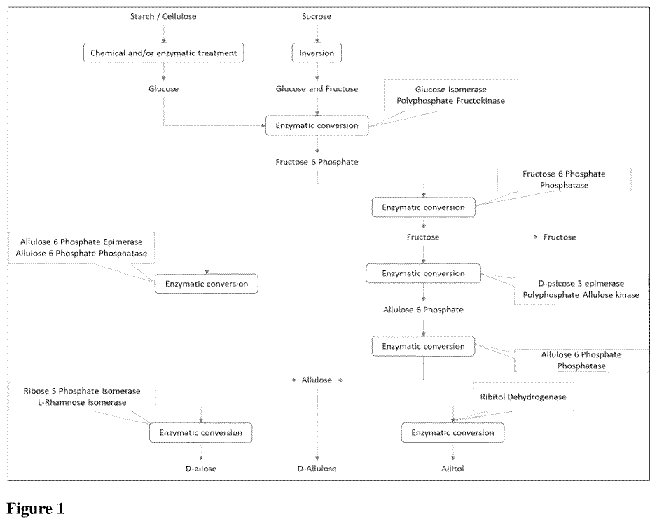

### DETAILED DESCRIPTION

At the very outset of the detailed description, it may be understood that the ensuing description only illustrates a particular form of this invention. However, such a particular form is only exemplary embodiment, and without intending to imply any limitation on the scope of this invention. Accordingly, the description is to be understood as an exemplary embodiment and teaching of invention and not intended to be taken restrictively.

Unless defined otherwise, technical and scientific terms used herein have the same meaning as commonly understood by one of ordinary skill in the art to which this invention belongs. Further, unless otherwise required by context, singular terms shall include pluralities and plural terms shall include the singular.

The invention provides enzymatic pathways, or processes, for synthesizing allulose with a high product yield, while greatly decreasing the product separation costs and allulose production costs.

The inventors of the present invention have developed a novel, improved and simple process for the production of D-allulose and its derivatives from carbohydrate sources such as sucrose or fructose or glucose derived from starch and/or cellulose. The inventors of the present invention have devised a simple process for the production of D-allulose, fructose, allose and allitol wherein the claimed process overcomes the equilibrium limitations of conventional isomerization and epimerization processes and results in higher conversion efficiencies.

As opposed to conventional processes, the process according to the present invention avoids the use of large number of enzymes required for the production of D-allulose from fructose and the process does not require the utilization of expensive ATP and complex systems for its regeneration. In particular, multiple complex and expensive separation systems for sugars and other intermediates is avoided. The process of the present invention involves the use of polyphosphate as phosphate donor, in place of ATP or ADP which happens in-vivo. For utilization of polyphosphate as sole phosphate donor, the invention included modifications in glucokinase or fructokinase to increase the affinity to polyphosphate rather than ATP or ADP.

The process of the present invention demonstrates higher conversion rate of raw material to D-fructose. The raw materials used in the process of the present invention are selected from the sugars/sugar polysaccharides which can be chosen from a group that includes but not limited to starch or its derivatives, cellulose or its derivatives, and sucrose. Examples of starch derivatives include amylose, amylopectin, soluble starch, amylodextrin, maltodextrin, maltose, and glucose. In certain embodiments of the invention, the process for preparing allulose involves converting starch to a starch derivative using enzymes such as Glucan phosphorylase, pullulanase, amylase or a combination of these enzymes.

The present invention also provides methods of modification of enzymes towards improvement in thermostability, catalytic activity, change in substrate affinity by way of gene editing methods including but not limited to point mutation, domain swapping, truncations, and amino acid mutations. The modifications are based on bioinformatic analysis of gene and protein, as well as simulation studies.

A major aspect of the present invention is the development of fusion proteins expressing one or more enzymes for the conversion of carbohydrates to D-allulose. The preparation of such fusion proteins includes culturing cells engineered to express at least one α-glucan phosphorylase and/or at least one cellodextrin phosphorylase, at least one phosphoglucomutase, at least one phosphoglucoisomerase, at least one allulose 6-phosphate epimerase, at least one allulose 6-phosphate phosphatase, or a combination of at least two (e.g., at least three, or at least four) of the foregoing enzymes.

A fusion protein prepared by the present invention may be created by joining two or more gene or gene segments that code for separate proteins. Translation of this fusion gene results in a single or multiple polypeptides with functional properties derived from each of the original proteins. A polyfunctional protein is a single protein that has at least two different activities, wherein that functionality is a native biological function or the result of an engineered enzyme fusion. Other enzymes may also be expressed as a single fusion protein or a polyfunctional protein. Thus, a fusion protein may contain multiple functionalities of any of the pathway enzymes described herein.

Furthermore, the fusion enzymes of the present invention are generated at cloning/sub-cloning of the gene construct, using either fixed linker or rigid linker between the two genes, and expression as a fusion protein under control of single promoter.

### Embodiments of the Present Invention

Accordingly, an embodiment of the present invention is to provide a process for the production of D-allulose and its derivatives, wherein the method comprises the steps of:
a. Converting the carbohydrate source to a mixture of glucose and fructose in the presence of extracellular enzymes selected from invertase, amylase and/or pullulanase;
b. Optionally converting the glucose in the mixture of step (a) to fructose through enzymatic conversion in the presence of isomerase,
c. Converting the fructose obtained in step (b) to fructose 6 phosphate in the presence of fructokinase,
d. Converting the fructose 6 phosphate produced in the step (c) to allulose 6-phosphate in the presence of allulose 6-phosphate 3-epimerase;
e. Converting the allulose 6-phosphate obtained in step (d) to D-allulose in the presence of enzyme allulose 6 phosphate phosphatase;
wherein the enzymes fructokinase, allulose 6-phosphate 3-epimerase and allulose 6 phosphate phosphatase are expressed by a part a single fusion protein.

An embodiment of the present invention is to provide an improved and simple process for the production of D-allulose and its derivatives, wherein the method comprises the steps of:
a. Converting the carbohydrate source to glucose using extracellular enzymes selected from invertase, amylase and/or pullulanase,
b. Converting the glucose produced in the step (a) in the presence of polyphosphate to glucose 6-phosphate by the enzyme glucokinase,
c. Converting the glucose 6-phosphate produced in step (b) to fructose 6 phosphate in the presence of phosphoglucoisomerase,
d. Converting the fructose 6 phosphate produced in the step (c) to allulose 6-phosphate in the presence of allulose 6-phosphate 3-epimerase;
e. Converting the allulose 6-phosphate obtained in step (d) to D-allulose in the presence of enzyme allulose 6 phosphate phosphatase;
wherein the allulose 6-phosphate 3-epimerase and allulose 6 phosphate phosphatase are expressed by a part a single fusion protein.

An embodiment of the present invention is to provide a process for the production of D-allulose and its derivatives from carbohydrate source, wherein the method comprises the steps of:
a. Converting the carbohydrate source to glucose-1 phosphate in the presence of glycogen/glucan phosphorylase;
b. converting the glucose-1 phosphate obtained in step (a) to glucose 6-phosphate in the presence of the enzyme phosphoglucomutase;
c. converting the glucose 6-phosphate produced in step (b) to fructose 6 phosphate in the presence of phosphoglucoisomerase,
d. Converting the fructose 6 phosphate produced in the step (c) to allulose 6-phosphate in the presence of allulose 6-phosphate 3-epimerase;
e. Converting the allulose 6-phosphate obtained in step (d) to D-allulose in the presence of enzyme allulose 6 phosphate phosphatase;
wherein the allulose 6-phosphate 3-epimerase and allulose 6 phosphate phosphatase are expressed by a part a single fusion protein.

An embodiment of the present invention is to provide a process for the production of D-allulose and its derivatives wherein, wherein the carbohydrate source is selected from sucrose, fructose, or glucose derived from starch and/or cellulose.

An embodiment of the present invention is to provide a process for the production of D-allulose and its derivatives, wherein the conversion efficiency is in the range of 75-95%.

An embodiment of the present invention is to provide a process for the production of D-allulose and its derivatives, wherein the enzymatic conversion of carbohydrate source is further followed by acid hydrolysis.

An embodiment of the present invention is to provide a process for the production of D-allulose and its derivatives, wherein enzymatic conversion of carbohydrate source is further followed by passage through strong cation exchange resins.

An embodiment of the present invention is to provide a process for the production of D-allulose and its derivatives, wherein the fusion enzymes are introduced in the reaction mixture in either free or immobilized form or combination thereof.

An embodiment of the present invention is to provide a process for the production of D-allulose and its derivatives, wherein the enzymatic conversions are carried out in the presence of inorganic polyphosphate or ATP.

Another important embodiment of the present invention, wherein the derivates of D-allulose are selected from D-allose and Allitol.

Yet another important embodiment of the present invention is to provide a process for the production of D-allose wherein the method comprises converting D-allulose obtained to D-allose using ribose-5-phosphate isomerase and or/L-rhamnose isomerase,

Yet another important embodiment of the present invention is to provide a process for the production of Allitol, wherein the method comprises converting D-allulose obtained to allitol using the enzyme ribitol dehydeogenase.

An embodiment of the present invention is to provide a process for the production of D-allulose and its derivatives, wherein the enzymes are used in free or immobilized form or combination thereof.

An embodiment of the present invention is to provide a process for the production of D-allulose and its derivatives, wherein D-allose or Allitol is produced using separate enzymes or multienzyme complex with more than one activity.

Yet another important embodiment of the present invention is to provide a process for the preparation of the fusion enzymes, the method comprising the steps of:
a. preparing recombinant cells for expressing the recombinant fusion proteins encoding the enzymes fructokinase, allulose 6-phosphate 3-epimerase and allulose 6 phosphate phosphatase;
b. culturing the recombinant cells at 37° C. followed by inducing fusion protein expression under different concentration of IPTG and at a temperature of 20-30° C.
c. extracting and isolating the fusion proteins from culture by causing cell lysis and after separation of soluble and insoluble fractions.

Yet another important embodiment of the present invention is to provide D-allulose and its derivatives obtained by the process of the present invention.

Yet another important embodiment of the present invention is to provide D-allulose and its derivates obtained by the process of the present invention for use as sugar substitutes.

### Advantages

1. Simple process for the production of D-allulose and its derivatives.

2. The number of enzymes required for the production of D-allulose to fructose is reduced.

3. Usage of enzymes as fusion enzymes which in turn further reduces process steps and hence, the process is faster compared to conventional processes.

4. The process does not require the utilization of expensive ATP and complex systems for its regeneration.

5. The requirement of complex and expensive simulated moving bed chromatographic separation systems is avoided.

6. Higher conversion rate of raw material to D-allulose is possible as the reaction equilibrium is overcome.

7. Enzymatic conversion with packed bed systems avoids the contamination of final product with proteins.

8. Packed systems offer high rate of reaction hence the reactor size is small reducing the capital expenses requirement.

9. Multistep process ensures that each enzyme used in the process is working at its optimal condition.

10. Immobilized enzymes have higher stability hence the requirement of frequent replenishment is minimized, provides benefit on operational cost reduction.

Without limiting the scope of the present invention as described above in any way, the present invention has been further explained through the examples provided below.

### Experimental Data

### Example 1

### Expression of Fructokinase, Allulose 6-Phosphate Epimerase and Allulose 6-Phosphate Phosphatase as a Single Fusion Enzyme

Enzymes meant for fructose conversion to Allulose including fructokinase, Allulose 6-phosphate epimerase and Allulose 6-phosphate phosphatase, were expressed as recombinant protein from E. coli BL 21 cells. Protein expression was under control of IPTG induction (T7lac promoter). Protein expression was induced by expression under different concentration of IPTG and at 25° C. (culture growth at 37° C.).

Protein expression from culture analysed by cell lysis and separation of soluble and insoluble fractions. All the separation fractions including crude lysate (C), soluble fraction(S) and pellet/insoluble fraction (P), were analysed in comparison with Protein molecular weight standard/ladder (L) and uninduced culture lysate (U) as negative control. Sample analysis was performed by electrophoresis on 12% polyacrylamide gel under standard conditions.

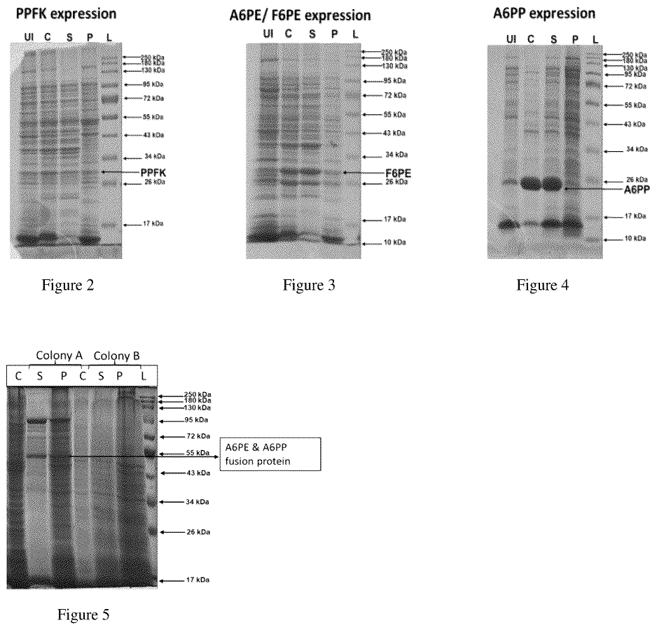

The FIGS. 2, 3 and 4 represent the SDS-PAGE analysis of fructokinase, Allulose 6-phosphate epimerase and Allulose 6-phosphate phosphatase.

### Example 2

### Expression of Allulose 6-Phosphate Epimerase and Allulose 6-Phosphate Phosphatase as a Single Fusion Enzyme

Enzymes meant for fructose conversion to Allulose including Allulose 6-phosphate epimerase and Allulose 6-phosphate phosphatase, were expressed as recombinant protein from E. coli BL 21 cells as a Fusion protein under control of single promoter induced by IPTG induction (T7lac promoter). Both the proteins were linked by flexible linker to get the fused expression of both protein together. Fusion Protein expression was induced by expression under different concentration of IPTG and at 25° C. (culture growth at 37° C.).

Fusion protein expression from culture analyzed by cell lysis and separation of soluble and insoluble fractions. All the separation fractions including crude lysate (C), soluble fraction(S) and pellet/insoluble fraction (P), from different colonies (colony A and B) were analyzed in comparison with Protein molecular weight standard/ladder (L). Sample analysis was performed by electrophoresis on 12% polyacrylamide gel under standard conditions.

FIG. 5 represent the SDS-PAGE analysis of Allulose 6-phosphate epimerase and Allulose 6-phosphate phosphatase Fusion protein.

### Example 3

### Preparation of D-Allulose From Carbohydrate Source Through the Use of Fusion Protein Expressing the Enzymes Fructokinase, Allulose 6-Phosphate 3-Epimerase and Allulose 6 Phosphate Phosphatase

In a reaction vessel a carbohydrate source selected from starch or cellulose is converted to a mixture of glucose and fructose in the presence of extracellular enzymes such as invertase. The glucose obtained is further enzymatically converted to fructose through catalytic reaction by the enzyme glucose isomerase.

Addition of a fusion protein expressing the enzymes fructokinase, allulose 6-phosphate 3-epimerase and allulose 6 phosphate phosphatase to the reaction mixture containing fructose. The fructose is converted to fructose 6 phosphate in the presence of fructokinase, and subsequently converting the fructose 6 phosphate to allulose 6-phosphate in the presence of allulose 6-phosphate 3-epimerase. Finally, the allulose 6-phosphate is converted to D-allulose in the presence of enzyme allulose 6 phosphate phosphatase.

### Example 4

### Preparation of D-Allulose From Carbohydrate Source Through the Use of Fusion Protein Expressing the Enzymes Allulose 6-Phosphate 3-Epimerase and Allulose 6 Phosphate Phosphatase

In a reaction vessel a carbohydrate source selected from starch or cellulose is converted to a mixture of glucose in the presence of extracellular enzymes such as invertase. The glucose obtained is further enzymatically converted to in the presence of polyphosphate to glucose 6-phosphate by the enzyme glucokinase which is subsequently converted to fructose 6 phosphate in the presence of phosphoglucoisomerase.

Addition of a fusion protein expressing the enzymes allulose 6-phosphate 3-epimerase and allulose 6 phosphate phosphatase to the reaction mixture containing fructose 6 phosphate. The fructose 6 phosphate is converted to allulose 6-phosphate in the presence of allulose 6-phosphate 3-epimerase. Finally, the allulose 6-phosphate is converted to D-allulose in the presence of enzyme allulose 6 phosphate phosphatase.

### Example 5

### Preparation of D-Allulose From Carbohydrate Source Through the Use of Fusion Protein Expressing the Enzymes Allulose 6-Phosphate 3-Epimerase and Allulose 6 Phosphate Phosphatase

In a reaction vessel a carbohydrate source selected from starch or cellulose is converted to a mixture of glucose-1 phosphate in the presence of glycogen/glucan phosphorylase. The glucose-1 phosphate obtained in step (a) to glucose 6-phosphate in the presence of the enzyme phosphoglucomutase which is subsequently converted to fructose 6 phosphate in the presence of phosphoglucoisomerase.

Addition of a fusion protein expressing the enzymes allulose 6-phosphate 3-epimerase and allulose 6 phosphate phosphatase to the reaction mixture containing fructose 6 phosphate. The fructose 6 phosphate is converted to allulose 6-phosphate in the presence of allulose 6-phosphate 3-epimerase. Finally, the allulose 6-phosphate is converted to D-allulose in the presence of enzyme allulose 6 phosphate phosphatase.

### Example 6

### Enzymatic Synthesis of D-Allose From D-allulose

In a reaction vessel, the D-allulose obtained from either example 3-5 are reacted further for enzymatically conversion to D-allose using the enzymes selected from ribose-5-phosphate isomerase and or/L-rhamnose isomerase.

### Example 7

### Enzymatic Synthesis of Allitol From D-Allulose

In a reaction vessel, the D-allulose obtained from either example 3-5 are reacted further for enzymatically conversion to allitol in the presence of the enzyme ribitol dehydeogenase.

The foregoing broadly outlines the features and technical advantages of the present disclosure in order that the detailed description of the disclosure that follows may be better understood. It should be appreciated by those skilled in the art that the conception and specific embodiment disclosed may be readily utilized as a basis for modifying the disclosed methods or for carrying out the same purposes of the present disclosure.

It must be noted that as used herein, the singular forms “a”, “an”, and “the”, include plural references unless the context clearly indicates otherwise. Thus, for example, reference to “an expression cassette” includes one or more of the expression cassettes disclosed herein and reference to “the method” includes reference to equivalent steps and methods known to those of ordinary skill in the art that could be modified or substituted for the methods described herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e. g., Sambrook et al., Molecular Cloning: A Laboratory Manual, 3rd ed., Cold Spring Harbor Laboratory Press, Cold Spring Harbor, N. Y. (2001); Ausubel et al., Current Protocols in Molecular Biology, J, Greene Publishing Associates (1992, and Supplements to 2002); Handbook of Biochemistry: Section A Proteins, Vol I 1976 CRC Press; Handbook of Biochemistry: Section A Proteins, Vol II 1976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

We claim:

1. A process for the production of D-allulose and its derivatives from carbohydrate source, wherein the method comprises the steps of:
a. Converting the carbohydrate source to a mixture of glucose and fructose in the presence of extracellular enzymes selected from invertase, amylase and/or pullulanase;
b. Optionally converting the glucose in the mixture of step (a) to fructose through enzymatic conversion in the presence of isomerase,
c. Converting the fructose from step (a) and/or step (b) to fructose 6 phosphate in the presence of fructokinase,
d. Converting the fructose 6 phosphate produced in the step (c) to allulose 6-phosphate in the presence of allulose 6-phosphate 3-epimerase;
e. Converting the allulose 6-phosphate obtained in step (d) to D-allulose in the presence of enzyme allulose 6 phosphate phosphatase;
wherein the enzymes fructokinase, allulose 6-phosphate 3-epimerase and allulose 6 phosphate phosphatase are expressed by a single fusion protein.

2. A process for the production of D-allulose and its derivatives from carbohydrate source, wherein the method comprises the steps of:
a. Converting the carbohydrate source to glucose using extracellular enzymes selected from invertase, amylase and/or pullulanase,
b. Converting the glucose produced in the step (a) in the presence of polyphosphate to glucose 6-phosphate by the enzyme glucokinase,
c. Converting the glucose 6-phosphate produced in step (b) to fructose 6 phosphate in the presence of phosphoglucoisomerase,
d. Converting the fructose 6 phosphate produced in the step (c) to allulose 6-phosphate in the presence of allulose 6-phosphate 3-epimerase;
e. Converting the allulose 6-phosphate obtained in step (d) to D-allulose in the presence of enzyme allulose 6 phosphate phosphatase;
wherein the allulose 6-phosphate 3-epimerase and allulose 6 phosphate phosphatase are expressed by a single fusion protein.

3. A process for the production of D-allulose and its derivatives from carbohydrate source, wherein the method comprises the steps of:
a. Converting the carbohydrate source to glucose-1 phosphate in the presence of glycogen/glucan phosphorylase;
b. converting the glucose-1 phosphate obtained in step (a) to glucose 6-phosphate in the presence of the enzyme phosphoglucomutase;
c. converting the glucose 6-phosphate produced in step (b) to fructose 6 phosphate in the presence of phosphoglucoisomerase,
d. Converting the fructose 6 phosphate produced in the step (c) to allulose 6-phosphate in the presence of allulose 6-phosphate 3-epimerase;
e. Converting the allulose 6-phosphate obtained in step (d) to D-allulose in the presence of enzyme allulose 6 phosphate phosphatase;
wherein the allulose 6-phosphate 3-epimerase and allulose 6 phosphate phosphatase are expressed by a single fusion protein.

4. The process as claimed in claim 1-3, wherein the carbohydrate source is selected from sucrose, fructose, or glucose derived from starch and/or cellulose.

5. The process as claimed in claim 1-3, wherein the fusion enzymes are introduced in the reaction mixture in either free or immobilized form or combination thereof.

6. The process as claimed in claims 1-3, wherein the conversion efficiency is in the range of 75-95%

7. The process as claimed in claim 1-3, wherein the enzymatic conversion of the carbohydrate source is further followed by acid hydrolysis.

8. The process as claimed in claim 1-3, wherein enzymatic conversion of the carbohydrate source is further followed by passage through strong cation exchange resins.

9. The process as claimed in claimed in claim 1, wherein the enzymes reaction is carried out in the presence of inorganic polyphosphate or ATP.

10. The process as claimed in claim 1-3, wherein the derivates of D-allulose are selected from D-allose and Allitol.

11. The process as claimed in claim 10, further comprising the steps for producing D-allose wherein the method comprises converting D-allulose obtained in step (d) of claim 1-3 to D-allose using ribose-5-phosphate isomerase and or/L-rhamnose isomerase,

12. The process as claimed in claim 10, further comprising the steps for producing Allitol, wherein the method comprises converting D-allulose obtained in step (d) of claim 1-3 to allitol using the enzyme ribitol dehydeogenase.

13. A process for the preparation of the fusion enzymes, the method comprising the steps of:
a. preparing recombinant cells for expressing the recombinant fusion proteins encoding the enzymes fructokinase, allulose 6-phosphate 3-epimerase and allulose 6 phosphate phosphatase;
b. culturing the recombinant cells at 37° C. followed by inducing fusion protein expression under different concentration of IPTG and at a temperature of 20-30° C.
c. extracting and isolating the fusion proteins from culture by causing cell lysis and after separation of soluble and insoluble fractions.

14. D-allulose and its derivates produced by the process as claimed in claims 1-12.

15. D-allulose and its derivates as obtained by the process as claimed in claim 1-12 for use as sugar substitutes.
